SEQUENCE LISTING (4 sequences)

SEQ ID NO: 1 - protein, 577 aa, Artificial Sequence
  Synthetic TBL1X isoform A
MTELAGASSSCCHRPAGRGAMQSVLHHFQRLRGREGGSHFINTSSPRGEAKMSITSDEVNFLVYRYLQESGFSHSAFTFGIESHISQSNINGTLVPPAALISILQKGLQYVEAEISINEDGTVFDGRPIESLSLIDAVMPDVVQTRQQAFREKLAQQQASAAAAAAAATAAATAATTTSAGVSHQNPSKNREATVNGEENRAHSVNNHAKPMEIDGEVEIPSSKATVLRGHESEVFICAWNPVSDLLASGSGDSTARIWNLNENSNGGSTQLVLRHCIREGGHDVPSNKDVTSLDWNTNGTLLATGSYDGFARIWTEDGNLASTLGQHKGPIFALKWNRKGNYILSAGVDKTTIIWDAHTGEAKQQFPFHSAPALDVDWQNNTTFASCSTDMCIHVCRLGCDRPVKTFQGHTNEVNAIKWDPSGMLLASCSDDMTLKIWSMKQEVCIHDLQAHNKEIYTIKWSPTGPATSNPNSNIMLASASFDSTVRLWDIERGVCTHTLTKHQEPVYSVAFSPDGKYLASGSFDKCVHIWNTQSGNLVHSYRGTGGIFEVCWNARGDKVGASASDGSVCVLDLRK

SEQ ID NO: 2 - protein, 526 aa, Artificial Sequence
  Synthetic TBL1X isoform B
MSITSDEVNFLVYRYLQESGFSHSAFTFGIESHISQSNINGTLVPPAALISILQKGLQYVEAEISINEDGTVFDGRPIESLSLIDAVMPDVVQTRQQAFREKLAQQQASAAAAAAAATAAATAATTTSAGVSHQNPSKNREATVNGEENRAHSVNNHAKPMEIDGEVEIPSSKATVLRGHESEVFICAWNPVSDLLASGSGDSTARIWNLNENSNGGSTQLVLRHCIREGGHDVPSNKDVTSLDWNTNGTLLATGSYDGFARIWTEDGNLASTLGQHKGPIFALKWNRKGNYILSAGVDKTTIIWDAHTGEAKQQFPFHSAPALDVDWQNNTTFASCSTDMCIHVCRLGCDRPVKTFQGHTNEVNAIKWDPSGMLLASCSDDMTLKIWSMKQEVCIHDLQAHNKEIYTIKWSPTGPATSNPNSNIMLASASFDSTVRLWDIERGVCTHTLTKHQEPVYSVAFSPDGKYLASGSFDKCVHIWNTQSGNLVHSYRGTGGIFEVCWNARGDKVGASASDGSVCVLDLRK

SEQ ID NO: 3 - protein, 522 aa, Artificial Sequence
  Synthetic TBL1Y
MSITSDEVNFLVYRYLQESGFSHSAFTFGIESHISQSNINGTLVPPSALISILQKGLQYVEAEISINKDGTVFDSRPIESLSLIVAVIPDVVQMRQQAFGEKLTQQQASAAATEASAMAKAATMTPAAISQQNPPKNREATVNGEENGAHEINNHSKPMEIDGDVEIPPNKATVLRGHESEVFICAWNPVSDLLASGSGDSTARIWNLNENSNGGSTQLVLRHCIREGGHDVPSNKDVTSLDWNSDGTLLAMGSYDGFARIWTENGNLASTLGQHKGPIFALKWNKKGNYVLSAGVDKTTIIWDAHTGEAKQQFPFHSAPALDVDWQNNMTFASCSTDMCIHVCRLGCDHPVKTFQGHTNEVNAIKWDPSGMLLASCSDDMTLKIWSMKQDACVHDLQAHSKEIYTIKWSPTGPATSNPNSSIMLASASFDSTVRLWDVEQGVCTHTLMKHQEPVYSVAFSPDGKYLASGSFDKYVHIWNTQSGSLVHSYQGTGGIFEVCWNARGDKVGASASDGSVCVLDL

SEQ ID NO: 4 - protein, 514 aa, Artificial Sequence
  Synthetic TBL1XR1
MSISSDEVNFLVYRYLQESGFSHSAFTFGIESHISQSNINGALVPPAALISIIQKGLQYVEAEVSINEDGTLFDGRPIESLSLIDAVMPDVVQTRQQAYRDKLAQQQAAAAAAAAAAASQQGSAKNGENTANGEENGAHTIANNHTDMMEVDGDVEIPPNKAVVLRGHESEVFICAWNPVSDLLASGSGDSTARIWNLSENSTSGSTQLVLRHCIREGGQDVPSNKDVTSLDWNSEGTLLATGSYDGFARIWTKDGNLASTLGQHKGPIFALKWNKKGNFILSAGVDKTTIIWDAHTGEAKQQFPFHSAPALDVDWQSNNTFASCSTDMCIHVCKLGQDRPIKTFQGHTNEVNAIKWDPTGNLLASCSDDMTLKIWSMKQDNCVHDLQAHNKEIYTIKWSPTGPGTNNPNANLMLASASFDSTVRLWDVDRGICIHTLTKHQEPVYSVAFSPDGRYLASGSFDKCVHIWNTQTGALVHSYRGTGGIFEVCWNAAGDKVGASASDGSVCVLDLRK

(19) United States
(12) Patent Application Publication
(10) Pub. No.: US 2013/0123281 A1
(43) Pub. Date: May 16, 2013

(54) Compositions and Methods for Inhibition of TBL-1 Binding to Disease-Associated Molecules

(71) Applicants: Hariprasad M. Vankayalapati, Draper, UT (US); Stephen Horrigan, Gaithersburg, MD (US)

(72) Inventors: Hariprasad M. Vankayalapati, Draper, UT (US); Stephen Horrigan, Gaithersburg, MD (US)

(73) Assignee: BETA CAT PHARMACEUTICALS, LLC, Gaithersburg, MC (US)

(21) Appl. No.: 13/674,638

(22) Filed: Nov. 12, 2012

Related U.S. Application Data
(60) Provisional application No. 61/558,823, filed on Nov. 11, 2011.

(51) Int. Cl. C07D 403/14; C07D 401/12; C07D 401/04; C07D 401/14; C07D 403/12; C07D 405/14; C07D 473/34; C07D 403/04

(52) U.S. Cl. CPC C07D 403/14; C07D 473/34; C07D 401/12; C07D 403/04; C07D 401/14; C07D 403/12; C07D 405/14; C07D 401/04
USPC 514/263.2; 514/275; 514/269; 514/256; 546/188; 514/341; 544/331; 544/319; 544/328; 544/277; 546/275.4; 435/7.92; 506/8; 435/7.94

(57) ABSTRACT

Compositions and methods which modulate diseases and disorders related to transducin β-like protein 1 (TBL1) activity, including but not limited to cancer, inflammation, and bone related diseases.

### FIELD OF THE INVENTION

The present invention relates to the field of therapeutic methods, compositions, processes and uses thereof to modulate diseases and disorders related to transducin β-like protein 1 (TBL1) activity, including but not limited to cancer, inflammation, and bone related diseases.

### BACKGROUND OF THE INVENTION

Cancer is the second leading cause of death in the United States. It presents complex challenges for the development of new therapies. Cancer is characterized by the abnormal growth of malignant cells that have undergone a series of genetic changes that lead to growth of tumor mass and metastatic properties.

Transducin β-like protein 1 (TBL1) family of proteins has been shown to be involved in the transcriptional activator by acting as a co-regulator exchange factor. The TBL1 family is composed of TBL1X, TBL1Y and TBLR1 proteins. These proteins are components of the SMRT-nuclear receptor/co-repressor (N-CoR) complex where they act to exchange the co-repressors and co-activators on the complex. SMRT and NCoR are large co-repressor proteins that are involved in the transcriptional repression by many different nuclear receptors. TBL1 family of proteins forms a reversible complex with NCoR/SMRT to act as a transcriptional activator for nuclear receptors.

Beta-catenin (β-catenin) is part of a complex of proteins that constitute adherens junctions (AJs). AJs are necessary for the creation and maintenance of epithelial cell layers by regulating cell growth and adhesion between cells. β-catenin also anchors the actin cytoskeleton and may be responsible for transmitting the contact inhibition signal that causes cells to stop dividing once the epithelial sheet is complete.

Wnt/β-catenin pathway has been shown to play a role in cancer. Recent studies have shown that TBL1 is able to bind to β-catenin and recruit the complex to Wnt responsive promoters to activate specific transcriptional program. It has also been shown that TBL1 is required for β-catenin to actively transcribe target genes. Further, TBL1 appears to protect β-catenin from ubiquitination (a post-translational modification by certain enzymes) and degradation. However, the mechanism of the interaction between TBL1 and β-catenin is unknown.

Aberrant β-catenin signaling plays a important role in tumorigenesis. In particular, colorectal cancer is estimated to have greater than 80% mutations in the β-catenin pathway, leading to unregulated oncogenic signaling. Aberrant β-catenin signaling has been shown to be involved in various cancer types, including melanoma, breast, lung, liver, gastric, myeloma, and acute myeloid leukemia (AML). Further, aberrant Wnt/β-catenin signaling has been found in a large number of other disorders, including osteoporosis, osteoarthritis, polycystic kidney disease, diabetes, schizophrenia, vascular disease, cardiac disease, hyperproliferative disorders, and neurodegenerative diseases.

Accordingly, there is a need for agents that are able interrupt the Wnt/β-catenin pathway and inhibit the deregulated activity of this pathway for the treatment, diagnosis and prevention of β-catenin pathway-related disorders and diseases.

### SUMMARY OF THE INVENTION

The present invention provides methods and compositions for treating disease or disorders by inhibiting transducin β-like protein 1 (TBL1) from binding disease-associated molecules. In particular, the provided methods and compositions relate to the treatment, diagnosis, and/or prevention of β-catenin signaling pathway disorders. The invention also provides methods for screening for drugs that can be used to treat disease or disorders.

In one aspect, the present invention is directed to a method of treating and/or preventing a β-catenin related disorder comprising administering to a patient in need thereof a therapeutically effective amount of an agent that binds to a lateral groove of transducin β-like protein 1 (TBL1) protein having the sequence selected from the group consisting of SEQ ID NO: 1-4, thereby preventing binding of β-catenin to said lateral groove.

In a preferred embodiment, the lateral groove of TBL1 protein is defined by residues 32 to 57 of SEQ ID NO: 1.

In a preferred embodiment, the agent is selected from the group consisting of a small molecule, a peptide, or a mimetic, wherein said small molecule has a molecular weight of no more than 1000 Daltons.

In another preferred embodiment, the β-catenin related disorder includes cancer, including but not limited to, colon cancer, myeloid leukemia, and multiple myeloma.

In another embodiment, the invention provides an agent for treating and/or preventing a β-catenin related disorder, wherein said agent upon administration to a patient in need thereof binds to a lateral groove of TBL1 protein having the SEQ ID NO: 1, thereby preventing binding of β-catenin to said lateral groove.

In a preferred embodiment, the provided agent has the following structure:

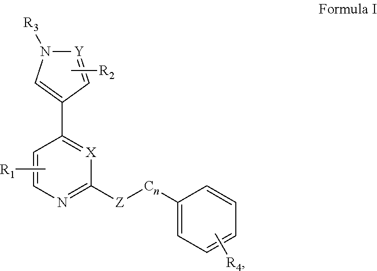

wherein
X and Y are selected from N and C;
R₁ is selected from H, halogen, CF₃, OCF₃, CN, SO₂CH₃, (CH₃)₂CN, SO₂NH₂, SO₂NCH₃, SO₂(CH₃)₂, SON(CH₃)₂, and C═ON(CH₃)₂;
R₂ is selected from H, CH₃, CH₂CH₃, F, and Cl;
R₃ is selected from H, —CH₃, C₁-C₆ alkyl, C₁-C₆ cycloalkyl, and C₁-C₆ hetero-cycloalkyl;
R₄ is selected from H, halo, OCH₃, CH₃, OH, NH₂, C₁-C₆ cycloalkyl, and C₁-C₆ hetero-cycloalkyl;
R₅ is selected from —CH₃ and halo;
Z is selected from —NH, O, S, N—CH₃, —NCH₂CH₃, and SO₂; and
n is 1 or 2;
or a pharmaceutically acceptable salt thereof.

In one embodiment, R₄ is selected from one of the following:

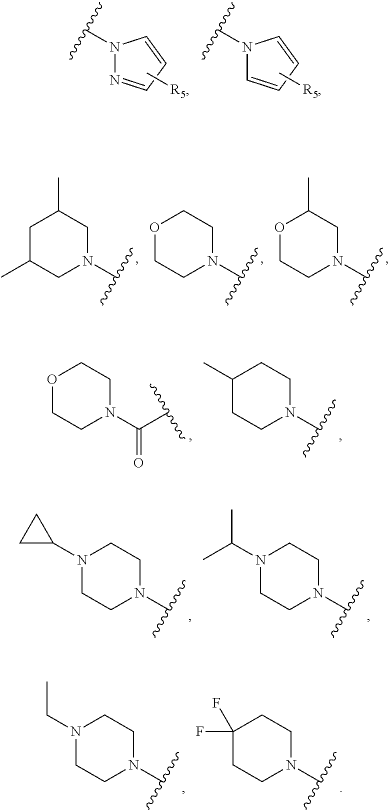

In another preferred embodiment, the provided agent has the following structure:

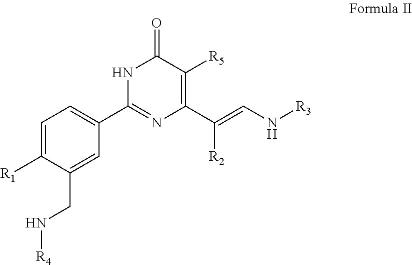

wherein
X is selected from N and C;
R₁ is selected from H, halogen, CF₃, OCF₃, CN, SO₂CH₃, (CH₃)₂ON, SO₂NH₂, SO₂NCH₃, SO₂(CH₃)₂, SON(CH₃)₂, and C═ON(CH₃)₂;
R₂ is selected from H, CH₃, CH₂CH₃, F, and Cl;
R₃ is selected from H and C₁-C₆ alkyl, Ar and Ar¹;

wherein Ar is phenyl substituted with 0-3 groups independently selected from cyano, C₁-C₆ alkyl, C₁-C⁶ haloalkyoxy, C₁-C₆ haloalkyl, C₁-C₆ polyhaloalkyl, C₁-C₆ cyanoalkyl, SO₂CH₃, SO₂CH₂CH₃, SO₂N(CH₃)₂, SO₂NH₂, SO₂NH—CH₃, SO₂—NHCF₃, SO₂NHCH₂CF₃, C₁-C₃ alkyl, C₁-C₃ alkylamine, and C₁-C₃ dialkylamino,

wherein Ar¹ is monocyclic heteroaryl substituted with 0-3 groups independently selected from halo, cyano, C₁-C₆ alkyl, C₁-C₆ haloalkyoxy, C₁-C₆ haloalkyl, and C₁-C₆ polyhaloalkyl, C₁-C₆ cyanoalkyl, SO₂CH₃, SO₂CH₂CH₃, SO₂N(CH₃)₂, SO₂NH₂, SO₂NH—CH₃, SO₂—NHCF₃, SO₂NHCH₂CF₃, C₁-C₃ alkyl, C₁-C₃ alkylamine, and C₁-C₃ dialkylamino;

R₄ is selected from H, CH₃, C═OCH₃, C═OCH₂—NH₂, C═OCH₂CH₃, C═OCH═CH₂, C═OCH═CH₂, C═OPr-i, and C═OCH₂OH; and
R₅ is selected from H, CH₃, and halogen,
or a pharmaceutically acceptable salt thereof.

In yet another preferred embodiment, the provided agent has the following structure:

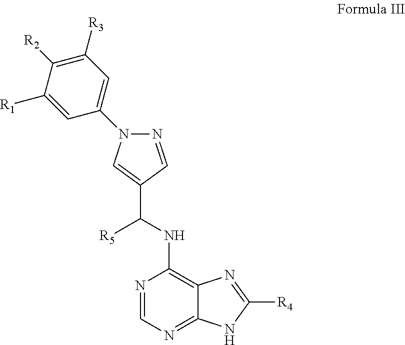

wherein
R₁ and R₃ are independently selected from hydrogen, halogen, and C₁-C₆ alkyl;
R₂ is a five-membered or six-membered C₃-C₆ heterocycle substituted with 0-3 groups selected from halogen, cyano, C₁-C₆ alkyl, C₁-C₆ haloalkyoxy, C₁-C₆ haloalkyl, and C₁-C₆ polyhaloalkyl;
R₄ is H, CH₃, halogen, CF₃, OCF₃, CN and —OCH₃;
R₅ is selected from H, CH₃, and halogen,
or a pharmaceutically acceptable salt thereof.

In the most preferred embodiment, the provided agent has the following structure:

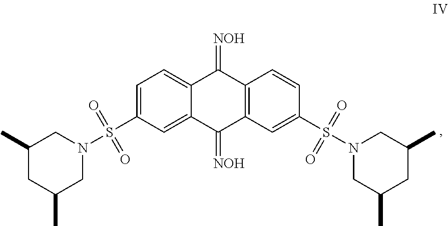

or a pharmaceutically acceptable salt thereof. This compound is referred to as “compound 1” throughout the application.

The invention also provides pharmaceutical compositions comprising the agents of the invention and pharmaceutically acceptable excipients.

In another embodiment, the invention provides a method of identifying an agent capable of modulating TBL1 activity comprising:
a) contacting a biological sample comprising an activator of TBL1 with a test agent; and
b) determining whether said test agent inhibits binding of said activator of TBL1 to TBL1;
   wherein if said test agent inhibits said binding, then said test agent is capable of modulating TBL1 activity.

In a preferred embodiment, TBL1 is selected from the group consisting of transducin (beta)-like 1X-linked (TBL1X), transducin (beta)-like 1Y-linked (TBL1Y) and transducin (beta)-like R1-linked TBLR1 proteins.

In one embodiment, the inhibition of binding in step (b) is measured by determining physical association of TBL1 and the activator.

In another embodiment, the inhibition of binding in step (b) is measured by determining the level or the stability of the activator.

In one embodiment, the activator is beta-catenin.

In another embodiment, the activator is a beta-catenin related protein.

In one embodiment, the method of identifying an agent capable of modulating TBL1 activity further comprises step (c) of determining whether said test agent binds to a TBL1 lateral groove.

In another embodiment, the invention provides a method of identifying an agent capable of modulating TBL1 activity comprising conducting a virtual screening of a library of test compounds, whereby said virtual screening is capable of predicting whether a test compound is able to bind to TBL1, wherein a test compound which is able to bind to TBL1 is identified as an agent capable of modulating a TBL1 activity.

The library can be either a physical library of small molecules and peptides or a virtual library of small molecules and peptides.

In one embodiment, the virtual screening is capable of predicting whether a test compound is able to form a hydrogen bond at position 35 of TBL1X.

### BRIEF DESCRIPTION OF THE DRAWINGS

FIG. 1 depicts a structure of the binding site of TBL1 with Compound 1 docked into lateral pocket;

FIG. 2 is a Western blot of an immunoprecipitation of HCT15 using TBL1 antibody;

FIG. 3 depicts a structure of the binding site of TBL1 with a prophetic compound;

FIG. 4 depicts a structure of the binding site of TBL1 with the best fit binding area designated in white; and

FIG. 5 depicts a structure of the best fit compound identified in the virtual screen docked into the binding site of TBL1.

### DETAILED DESCRIPTION OF THE INVENTION

### Definitions

The following definitions are used, unless otherwise described.

The term “prodrugs” refers to compounds, including but not limited to monomers and dimers of the compounds of the invention, which become under physiological conditions compounds of the invention or the active moieties of the compounds of the invention.

The term “active moieties” refers to compounds which are pharmaceutically active in vivo, whether or not such compounds are compounds of the invention.

The term “alkyl” refers to a monovalent saturated aliphatic hydrocarbon including straight chain and branched chain groups. Preferably, the alkyl group has 1 to 20 carbon atoms. More preferably, it is a medium alkyl (having 1 to 10 carbon atoms). Most preferably, it is a lower alkyl (having 1 to 4 carbon atoms). The alkyl group may be substituted or unsubstituted.

The term “alkoxy” group refers to both an —O-alkyl and an —O-cycloalkyl group; preferably an alkoxy group refers to a lower alkoxy, and most preferably methoxy or ethoxy.

The term “aryl” refers to a monocyclic or bicyclic aromatic group (e.g., phenyl or naphthyl) that can be unsubstituted or substituted, for example, with one or more, and in particular one to three, substituents, such as halo, alkyl, hydroxy, hydroxyalkyl, alkoxy, alkoxyalkyl, haloalkyl, nitro, amino, alkylamino, acylamino, alkylthio, alkylsulfonyl, and alkylsulfonyl.

The term “heteroaryl” refers to a monocyclic, bicyclic, or tricyclic ring system containing one, two, or three aromatic rings and containing at least one nitrogen, oxygen, or sulfur atom in an aromatic ring, and which can be unsubstituted or substituted, for example, with one or more, and in particular one to three, substituents, such as halo, alkyl, hydroxy, hydroxyalkyl, alkoxy, alkoxyalkyl, haloalkyl, nitro, amino, alkylamino, acylamino, alkylthio, alkylsulfonyl, and alkylsulfonyl. Examples of heteroaryl groups include, but are not limited to, 2H-pyrrolyl, 3H-indolyl, 4H-quinolizinyl, 4H-carbazolyl, acridinyl, benzo[b]thienyl, benzothiazolyl, 13-carbolinyl, carbazolyl, chromenyl, cinnaolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, naptho[2,3-b], oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrimidinyl, pyrrolyl, quinazolinyl, quinolyl, quinoxalinyl, thiadiazolyl, thianthrenyl, thiazolyl, thienyl, triazolyl, and xanthenyl.

The term “phenyl” refers to a cyclic group of atoms with the formula C₆H₅ and which can be unsubstituted or substituted, for example, with one or more, and in particular one to three, substituents, such as halo, alkyl, hydroxy, hydroxyalkyl, alkoxy, alkoxyalkyl, haloalkyl, nitro, amino, alkylamino, acylamino, alkylthio, alkylsulfonyl, and alkylsulfonyl.

The term “composition” is intended to encompass a product comprising the specified ingredients in the specified amounts, as well as any product which results, directly or indirectly, from a combination of the specified ingredients in the specified amounts.

The term “subject” includes mammals, including humans. The terms “patient” and “subject” are used interchangeably.

In general, unless indicated otherwise, a chemical group referred to anywhere in the specification can be optionally substituted.

The term “therapeutically effective amount” means the amount of a compound that, when administered to a subject for treating a disease or disorder, is sufficient to effect such treatment for the disease or disorder. The “therapeutically effective amount” can vary depending on the variety of factors, including the compound, the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than required to achieve the desired therapeutic effect and to gradually increase the dosage until the desired effect is achieved.

In one embodiment, the terms “treating” or “treatment” refer to ameliorating the disease or disorder (i.e., arresting or reducing the development of the disease or at least one of the clinical symptoms thereof). In another embodiment, “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder, or even preventing the same.

### DESCRIPTION OF THE INVENTION

The present invention provides methods and compositions for treating disease or disorders by inhibiting transducin β-like protein 1 (TBL1) from binding disease-associated molecules. In particular, the provided methods and compositions relate to the treatment, diagnosis, and/or prevention of β-catenin signaling pathway disorders. The invention also provides methods for screening for drugs that can be used to treat disease or disorders.

In one aspect, the present invention is directed to a method of treating and/or preventing a β-catenin related disorder comprising administering to a patient in need thereof a therapeutically effective amount of an agent that binds to a lateral groove of transducin β-like protein 1 (TBL1) protein having the sequence selected from the group consisting of SEQ ID NO: 1-4, thereby preventing binding of β-catenin to said lateral groove.

In a preferred embodiment, the lateral groove of TBL1 protein is defined by residues 32 to 57 of SEQ ID NO: 1.

In a preferred embodiment, the agent is selected from the group consisting of a small molecule, a peptide, or a mimetic, wherein said small molecule has a molecular weight of no more than 1000 Daltons.

In another preferred embodiment, the β-catenin related disorder includes cancer, including but not limited to, colon cancer.

In another embodiment, the invention provides an agent for treating and/or preventing a β-catenin related disorder, wherein said agent upon administration to a patient in need thereof binds to a lateral groove of TBL1 protein having the SEQ ID NO: 1, thereby preventing binding of β-catenin to said lateral groove.

In a preferred embodiment, the provided agent has the following structure:

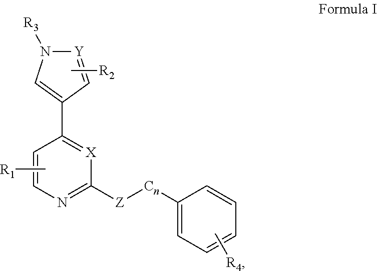

wherein
X and Y are selected from N and C;
R₁ is selected from H, halogen, CF₃, OCF₃, CN, SO₂CH₃, (CH₃)₂CN, SO₂NH₂, SO₂NCH₃, SO₂(CH₃)₂, SON(CH₃)₂, and C═ON(CH₃)₂;
R₂ is selected from H, CH₃, CH₂CH₃, F, and Cl;
R₃ is selected from H, —CH₃, C₁-C₆ alkyl, C₁-C₆ cycloalkyl, and C₁-C₆ hetero-cycloalkyl;
R₄ is selected from H, halo, OCH₃, CH₃, OH, NH₂, C₁-C₆ cycloalkyl, and C₁-C₆ hetero-cycloalkyl;
R₅ is selected from —CH₃ and halo;
Z is selected from —NH, O, S, N—CH₃, —NCH₂CH₃, and SO₂; and
n is 1 or 2;
or a pharmaceutically acceptable salt thereof.

In one embodiment, R₄ is selected from one of the following:

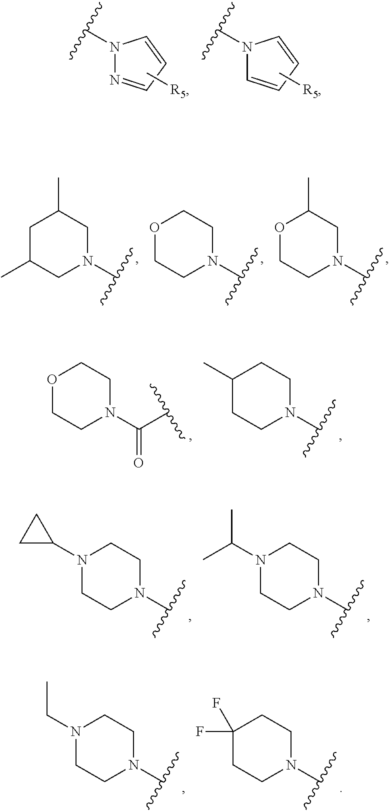

In another preferred embodiment, the provided agent has the following structure:

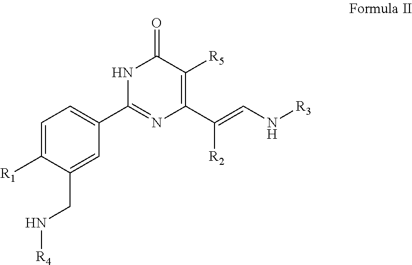

wherein
X is selected from N and C;
R₁ is selected from H, halogen, CF₃, OCF₃, CN, SO₂CH₃, (CH₃)₂CN, SO₂NH₂, SO₂NCH₃, SO₂(CH₃)₂, SON(CH₃)₂, and C═ON(CH₃)₂;
R₂ is selected from H, CH₃, CH₂CH₃, F, and Cl;
R₃ is selected from H and C₁-C₆ alkyl, Ar and Ar¹;

wherein Ar is phenyl substituted with 0-3 groups independently selected from cyano, C₁-C₆ alkyl, C₁-C⁶ haloalkyoxy, C₁-C₆ haloalkyl, C₁-C₆ polyhaloalkyl, C₁-C₆ cyanoalkyl, SO₂CH₃, SO₂CH₂CH₃, SO₂N(CH₃)₂, SO₂NH₂, SO₂NH—CH₃, SO₂—NHCF₃, SO₂NHCH₂CF₃, C₁-C₃ alkyl, C₁-C₃ alkylamine, and C₁-C₃ dialkylamino,

wherein Ar¹ is monocyclic heteroaryl substituted with 0-3 groups independently selected from halo, cyano, C₁-C₆ alkyl, C₁-C₆ haloalkyoxy, C₁-C₆ haloalkyl, and C₁-C₆ polyhaloalkyl, C₁-C₆ cyanoalkyl, SO₂CH₃, SO₂CH₂CH₃, SO₂N(CH₃)₂, SO₂NH₂, SO₂NH—CH₃, SO₂—NHCF₃, SO₂NHCH₂CF₃, C₁-C₃ alkyl, C₁-C₃ alkylamine, and C₁-C₃ dialkylamino;

R₄ is selected from H, CH₃, C═OCH₃, C═OCH₂—NH₂, C═OCH₂CH₃, C═OCH═CH₂, C═OCH═CH₂, C═OPr-i, and C═OCH₂OH; and
R₅ is selected from H, CH₃, and halogen,
or a pharmaceutically acceptable salt thereof.

In yet another preferred embodiment, the provided agent has the following structure:

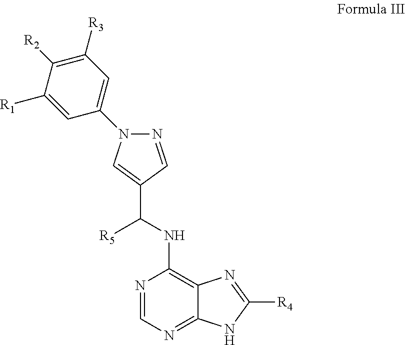

wherein
R₁ and R₃ are independently selected from hydrogen, halogen, and C₁-C₆ alkyl;
R₂ is a five-membered or six-membered C₃-C₆ heterocycle substituted with 0-3 groups selected from halogen, cyano, C₁-C₆ alkyl, C₁-C₆ haloalkyoxy, C₁-C₆ haloalkyl, and C₁-C₆ polyhaloalkyl;
R₄ is H, CH₃, halogen, CF₃, OCF₃, CN and —OCH₃;
R₅ is selected from H, CH₃, and halogen,
or a pharmaceutically acceptable salt thereof.

In the most preferred embodiment, the provided agent has the following structure:

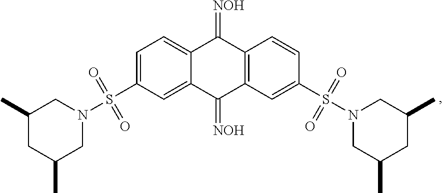

or a pharmaceutically acceptable salt thereof. This compound is referred to as “Compound 1” throughout the application

Compound 1 was originally identified in a cell based screen for its ability to inhibit the transcriptional activation or β-catenin genes. Characterization of this compound led to the discovery that the compound is able to induce the degradation of β-catenin, interfere with the transcriptional activation complex, and has characteristics of a nuclear receptor signaling pathway modulator. Both the target of Compound 1 and the exact mechanism of its action were unknown until the present invention.

Computational docking studies were undertaken to test the interaction of Compound 1 with TBL1. Using the mechanistic description of Compound 1 biologic activity, it was hypothesized that it might interact with TBL1 and prevent β-catenin from interacting and lead to its degradation.

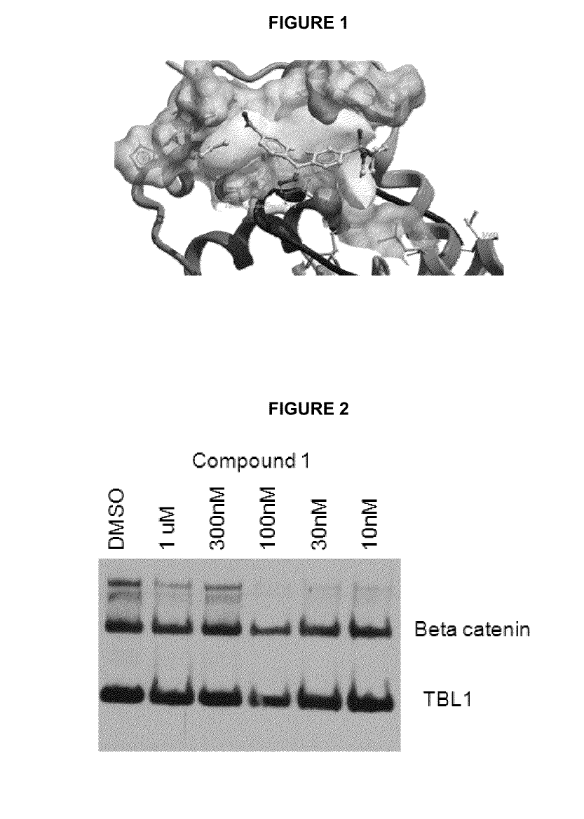

FIG. 1 describes a structure of the binding site of TBL1 with Compound 1 docked into lateral pocket of TBL1. Compound 1 was placed into a hydrophobic pocket of TBL1 defined by residues 32 to 57 of SEQ ID NO: 1 which is the amino acid sequence of TBL1. It was found that there was a specific interaction between Serine at position 35 of SEQ ID NO: 1 and Compound 1.

As Example 1 demonstrates, Compound 1 was found to disrupt the interaction of TBL1 with β-catenin in a cellular system.

The invention also provides methods for identifying modulators of TBL1 family members that can be used to select and test agents that inhibit the β-catenin signaling pathway and other related transcriptional co-activators that might bind into the same pocket. Thus, the invention allows the design of analogs of Compound 1 using computation docking studies, and the identification of new agents (which may or may not be analogs of Compound 1) that are able to interact within this binding pocket.

Thus, in one embodiment, the invention provides a method of identifying an agent capable of modulating TBL1 activity comprising:
a) contacting a biological sample comprising an activator of TBL1 with a test agent; and
b) determining whether said test agent inhibits binding of said activator of TBL1 to TBL1;
   wherein if said test agent inhibits said binding, then said test agent is capable of modulating TBL1 activity.

In a preferred embodiment, TBL1 is selected from the group consisting of transducin (beta)-like 1X-linked (TBL1X), transducin (beta)-like 1Y-linked (TBL1Y) and transducin (beta)-like R1-linked TBLR1 proteins.

The test agents can be obtained by any of the numerous methods known in the art including synthetic libraries, spatially addressed solid phase libraries, affinity selection of pooled libraries, synthetic peptide libraries, biological libraries including phage display technologies, or DNA and RNA aptamers.

In one embodiment, the assay can be a non-cellular assay with a TBL1 family member and purified β-catenin (or biologically portions thereof), either labeled or non-labeled with reporter molecules. The disruption of the binding of the two molecules can be measured by either direct measurement of physical association (e.g. SPR or acoustic detection) or by detecting changes in the level or the stability of the activator (e.g., an associated reporter molecule).

In another embodiment, the interaction of a TBL1 family member, either labeled or nonlabeled with reporter molecules, with compound I (or other compounds that bind to a lateral groove of TBL1 protein) can be measured. The disruption of the TBL1 family member and compound I interaction induced by test agents can be measured using either physical association (e.g. SPR or acoustic detection) or by detecting changes in the level or the stability of the activator (e.g., an associated reporter molecule).

In another embodiment, the interaction of TBL1 family member and β-catenin (or biologically portions thereof), can be measured in a cellular system using a reporter detection system that measures the ability of the two proteins to bind to one another, the stability of the β-catenin protein, or the transcriptional activity of the β-catenin protein. The cell can be of mammalian origin, or a yeast or bacterial cell.

In one embodiment, the activator is beta-catenin.

In another embodiment, the activator is a beta-catenin related protein.

In one embodiment, the method of identifying an agent capable of modulating TBL1 activity further comprises step (c) of determining whether said test agent binds to a TBL1 lateral groove.

In another embodiment, the invention provides a method of identifying an agent capable of modulating TBL1 activity comprising conducting a virtual screening of a library of test compounds, whereby said virtual screening is capable of predicting whether a test compound is able to bind to TBL1, wherein a test compound which is able to bind to TBL1 is identified as an agent capable of modulating a TBL1 activity.

The library can be either a physical library of small molecules and peptides or a virtual library of small molecules and peptides.

In one embodiment, the virtual screening is capable of predicting whether a test compound is able to form a hydrogen bond at position 35 of TBL1X.

In another embodiment, the invention provides a compound or agent obtainable using the described methods. Thus, the methods of the invention can be used to obtain a compound based on the structure and properties of compounds through interactive evaluation of both: 1) structural suitability to the computational docking model and 2) biologic activity in cell-based or non cell-based assays.

The compounds of this invention include pharmaceutically acceptable salts, enantiomers, stereoisomers, rotomers, tautomers, racemates and prodrugs of the compounds of the invention.

The compounds of the invention can exist in unsolvated as well as solvated forms, including hydrated forms, e.g., hemi-hydrate. In general, the solvated forms, with pharmaceutically acceptable solvents such as water, ethanol, and the like are equivalent to the unsolvated forms for the purposes of the invention.

The phrase “pharmaceutically acceptable salt” means those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, S. M. Berge et al. describe pharmaceutically acceptable salts in detail in J. Pharmaceutical Sciences, 1977, 66: 1 et seq.

Pharmaceutically acceptable salts include, but are not limited to, acid addition salts. For example, the nitrogen atoms may form salts with acids. Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methanesulfonate, nicotinate, 2-naphthalenesulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulfuric acid and phosphoric acid and such organic acids as oxalic acid, maleic acid, succinic acid and citric acid.

Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylammonium, dimethylammonium, trimethylammonium, triethylammonium, diethylammonium, and ethylammonium among others. Other representative organic amines useful for the formation of base addition salts include ethylenediamine, ethanolamine, diethanolamine, piperidine, piperazine and the like.

The present invention also provides pharmaceutical compositions that comprise compounds of the present invention formulated together with one or more non-toxic pharmaceutically acceptable carriers. The pharmaceutical compositions can be specially formulated for oral administration in solid or liquid form, for parenteral injection or for rectal administration.

The pharmaceutical compositions of this invention can be administered to humans and other mammals orally, rectally, parenterally, intracisternally, intravaginally, transdermally (e.g. using a patch), transmucosally, sublingually, pulmonary, intraperitoneally, topically (as by powders, ointments or drops), bucally or as an oral or nasal spray. The term “parenterally,” as used herein, refers to modes of administration which include intravenous, intramuscular, intraperitoneal, intrasternal, subcutaneous and intraarticular injection and infusion.

In another aspect, the present invention provides a pharmaceutical composition comprising a component of the present invention and a physiologically tolerable diluent. The present invention includes one or more compounds as described above formulated into compositions together with one or more non-toxic physiologically tolerable or acceptable diluents, carriers, adjuvants or vehicles that are collectively referred to herein as diluents, for parenteral injection, for intranasal delivery, for oral administration in solid or liquid form, for rectal or topical administration, among others.

Compositions suitable for parenteral injection may comprise physiologically acceptable, sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), vegetable oils (such as olive oil), injectable organic esters such as ethyl oleate, and suitable mixtures thereof.

These compositions can also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride and the like. Prolonged absorption of the injectable pharmaceutical form can be brought about by the use of agents delaying absorption, for example, aluminum monostearate and gelatin.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders and granules. In such solid dosage forms, the active compound may be mixed with at least one inert, pharmaceutically acceptable excipient or carrier, such as sodium citrate or dicalcium phosphate and/or a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

The active compounds can also be in micro-encapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof.

Besides inert diluents, the oral compositions may also include adjuvants such as wetting agents, emulsifying and suspending agents, sweetening, flavoring and perfuming agents.

Compositions for rectal or vaginal administration are preferably suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

Compounds of the present invention can also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients and the like. The preferred lipids are natural and synthetic phospholipids and phosphatidyl cholines (lecithins) used separately or together.

Methods to form liposomes are known in the art. See, for example, Prescott, Ed., Methods in Cell Biology, Volume XIV, Academic Press, New York, N.Y. (1976), p. 33 et seq.

Dosage forms for topical administration of a compound of this invention include powders, sprays, ointments and inhalants. The active compound is mixed under sterile conditions with a pharmaceutically acceptable carrier and any needed preservatives, buffers or propellants which can be required. Ophthalmic formulations, eye ointments, powders and solutions are also contemplated as being within the scope of this invention.

Actual dosage levels of active ingredients in the pharmaceutical compositions of this invention can be varied so as to obtain an amount of the active compound(s) which is effective to achieve the desired therapeutic response for a particular patient, compositions and mode of administration. The selected dosage level will depend upon the activity of the particular compound, the route of administration, the severity of the condition being treated and the condition and prior medical history of the patient being treated. However, it is within the skill of the art to start doses of the compound at levels lower than required to achieve the desired therapeutic effect and to gradually increase the dosage until the desired effect is achieved.

When used in the above or other treatments, a therapeutically effective amount of one of the compounds of the present invention can be employed in pure form or, where such forms exist, in pharmaceutically acceptable salt, ester or prodrug form. Alternatively, the compound can be administered as a pharmaceutical composition containing the compound of interest in combination with one or more pharmaceutically acceptable excipients.

The total daily dose of the compounds of this invention administered to a human or lower animal may range from about 0.0001 to about 1000 mg/kg/day. If desired, the effective daily dose can be divided into multiple doses for purposes of administration; consequently, single dose compositions may contain such amounts or submultiples thereof to make up the daily dose.

For a clearer understanding of the invention, details are provided below. These are merely illustrations and are not to be understood as limiting the scope of the invention in any way. Indeed, various modifications of the invention in addition to those shown and described herein will become apparent to those skilled in the art from the following examples and foregoing description. Such modifications are also intended to fall within the scope of the appended claims.

Structures of The Formula I-IV claimed structures are provided in the Tables 1-4

TABLE 1
4-1H-pyrazol-pyrimidin series inhibitors of TBL1 of Formula I

| Compound | Example | | | |
|---|---|---|---|---|
| No: | ID | Structure | Chemical Name | MW |
| 1 | 1 | 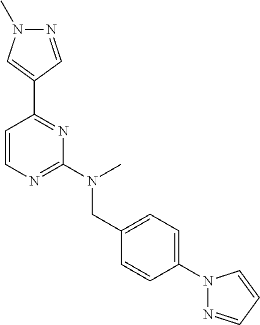 [SMILES: CN(Cc1ccc(-n2cccn2)cc1)c1nccc(-c2cnn(C)c2)n1] | N-(4-(1H-pyrazol-1- yl)benzyl)-N-methyl-4-(1- methyl-1H-pyrazol-4- yl)pyrimidin-2-amine | 345.40 |
| 2 | 2 | 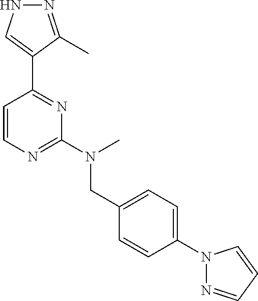 [SMILES: Cc1n[nH]cc1-c1ccnc(N(C)Cc2ccc(-n3cccn3)cc2)n1] | N-(4-(1H-pyrazol-1- yl)benzyl)-N-methyl-4-(3- methyl-1H-pyrazol-4- yl)pyrimidin-2-amine | 345.40 |
| 3 | 3 | 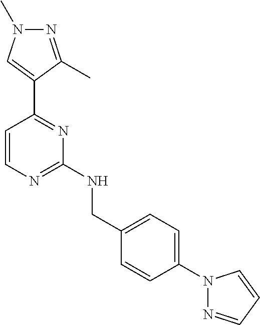 [SMILES: Cc1nn(C)cc1-c1ccnc(NCc2ccc(-n3cccn3)cc2)n1] | N-(4-(1H-pyrazol-1- yl)benzyl)-4-(1,3-dimethyl- 1H-pyrazol-4-yl)pyrimidin- 2-amine | 345.50 |
| 4 | 4 | 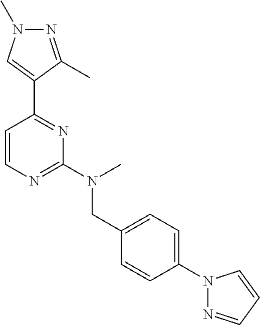 [SMILES: Cc1nn(C)cc1-c1ccnc(N(C)Cc2ccc(-n3cccn3)cc2)n1] | N-(4-(1H-pyrazol-1- yl)benzyl)-4-(1,3-dimethyl- 1H-pyrazol-4-yl)-N- methylpyridin-2-amine | 358.44 |
| 5 | 5 | 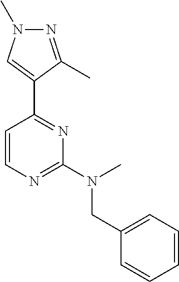 [SMILES: Cc1nn(C)cc1-c1ccnc(N(C)Cc2ccccc2)n1] | N-benzyl-4-(1,3-dimethyl- 1H-pyrazol-4-yl)-N- methylpyrimidin-2-amine | 293.37 |
| 6 | 6 | 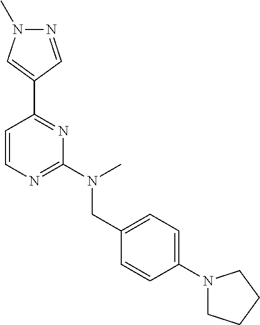 [SMILES: CN(Cc1ccc(N2CCCC2)cc1)c1nccc(-c2cnn(C)c2)n1] | N-methyl-4-(1-methyl-1H- pyrazol-4-yl)-N-(4- (pyrrolidin-1-yl)benzyl) pyrimidin-2-amine | 348.44 |
| 7 | 7 | 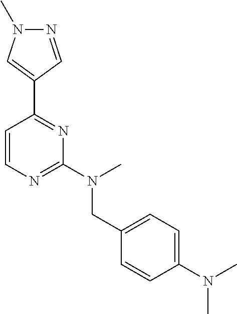 | N-(4-(dimethylamino)benzyl)- N-methyl-4-(1-methyl-1H- pyrazol-4-yl)pyrimidin-2- amine | 322.41 |
| 8 | 8 | 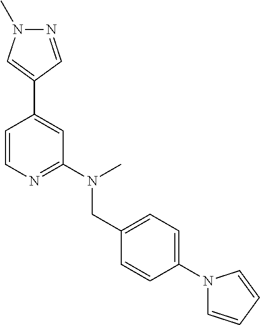 [SMILES: CN(Cc1ccc(-n2cccc2)cc1)c1cc(-c2cnn(C)c2)ccn1] | N-(4-(dimethylamino)benzyl)- N-methyl-4-(1-methyl-1H- pyrazol-4-yl)pyrimidin-2- amine | 343.42 |

TABLE 2
Lead 4-1H-pyrazol-pyrimidin series inhibitor of TBL1 of Formula I

| Compound | Example | | | |
|---|---|---|---|---|
| No: | ID | Structure | Chemical Name | MW |
| 9 | 29 | 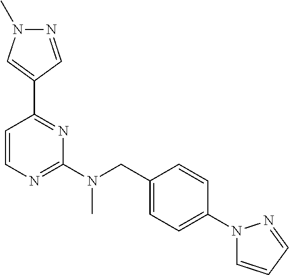 [SMILES: CN(Cc1ccc(-n2cccn2)cc1)c1nccc(-c2cnn(C)c2)n1] | N-(4-(1H-pyrazol-1-yl)benzyl)-3- (1,3-dimethyl-1H-pyrazol-4-yl)- N-methylaniline | 357.45 |

TABLE 3
Lead 2-phenyl-6-(pyridin-3-yl)pyrimidin-4(3H)-one series inhibitors of Formula II

| Compound | Example | | | |
|---|---|---|---|---|
| No: | ID | Structure | Chemical Name | MW |
| 10 | 16 | 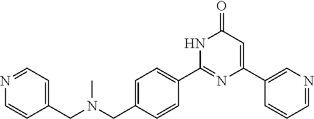 [SMILES: CN(Cc1ccncc1)Cc1ccc(-c2nc(-c3cccnc3)cc(=O)[nH]2)cc1] | 2-(4-((methyl(pyridin-4- ylmethyl)amino)methyl)phenyl)-6- (pyridin-3-yl)pyrimidin-4(3H)-one | 383.44 |
| 11 | 22 | 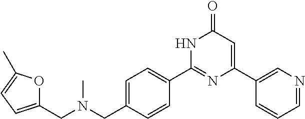 [SMILES: Cc1ccc(CN(C)Cc2ccc(-c3nc(-c4cccnc4)cc(=O)[nH]3)cc2)o1] | 2-(4-((methyl((5-methylfuran-2- yl)methyl)amino)methyl)phenyl)-6- pyridin-3-yl)pyrimidin-4(3H)-one | 386.44 |
| 12 | 34 | 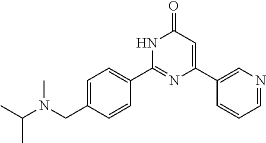 [SMILES: CC(C)N(C)Cc1ccc(-c2nc(-c3cccnc3)cc(=O)[nH]2)cc1] | 2-(4-((isopropyl(methyl)amino) methyl)phenyl)-6-(pyridin-3- yl)pyrimidin-4(3H)-one | 334.41 |
| 13 | 35 | 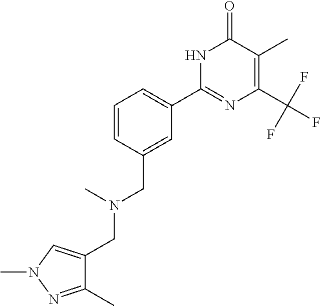 [SMILES: Cc1nn(C)cc1CN(C)Cc1cccc(-c2nc(C(F)(F)F)c(C)c(=O)[nH]2)c1] | 2-(4-((((1,3-dimethyl-1H-pyrazol- 4-yl)methyl)(methyl)amino) methyl)phenyl)-5-methyl-6- (trifluoromethyl)pyrimidin- 4(3H)-one | 405.41 |

TABLE 4
1H-pyrazol-4-yl)ethyl)-9H-purin-6-amine series of TBL1 inhibitors of Formula III

| Compound | Example | | | |
|---|---|---|---|---|
| No: | ID | Structure | Chemical Name | MW |
| 14 | 9 | 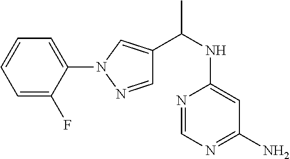 [SMILES: CC(Nc1cc(N)ncn1)c1cnn(-c2ccccc2F)c1] | N⁴-(1-(1-(2-fluorophenyl)-1H- pyrazol-4-yl)ethyl)pyrimidine- 4,6-diamine | 298.32 |
| 15 | 19 or 10 | 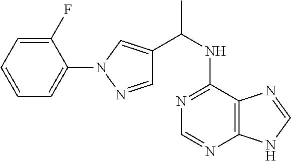 [SMILES: CC(Nc1ncnc2[nH]cnc12)c1cnn(-c2ccccc2F)c1] | N-(1-(1-(2-fluorophenyl)-1H- pyrazol-4-yl)ethyl)-9H-purin-6- amine | 323.33 |

### EXAMPLES

### Example 1

### Interaction of Compound 1 with β-Catenin and TBL1

A study was conducted where cells with activated β-catenin pathway were treated with an effective amount of Compound 1 and found that C was able to disrupt the interaction of β-catenin with TBL1 in a cancer cell.

FIG. 2 depicts Western blot of an immunoprecipitation using TBL1 antibody. HCT15 cells were incubated with DMSO or indicated concentration of compound for 6 hours. Cell lysates were made and immunoprecipitated with TBL1 antibody. Proteins were separated on an SDS gel, transferred to a membrane and probed with antibodies to β-catenin and to TBL1. Briefly, the HCT15 cell line was seeded at 70% confluence in medium containing 10% FBS and penicillin/streptomycin. After 24 h the cells were treated with Compound 1 for 6 h. Cell lysates were harvested, precleared with protein NG sepharose (Santa Cruz Biotech), and TBL1 was immunoprecipitated using mouse TBL1 antibody (Santa Cruz Biotech) conjugated to protein G sepharose (Santa Cruz Biotech). Immunoprecipitated proteins were resolved on a 10% SDS-PAGE gel followed by transfer of proteins to a nitrocellulose membrane. Western blot analysis was done using antibodies to detect β-catenin protein (BD Transduction Laboratories) and TBL1 protein (Santa Cruz Biotech).

Additionally the ability of test agents to modulate the interaction between TBL1 and beta catenin can be measured using a plate based assay that is suitable for screening for the activity and potency of multiple test agents.

HCT 15 cells are incubated with 20 mM LiCl to induce the Wnt pathway. After 24 h fresh medium with 20 mM LiCl and test agent is added at concentration in the range of 0.3 nM to 3 μM for 6 hours at 37° C. Protein lysates are prepared from cells lysed in RIPA buffer containing protease inhibitors, High affinity binding Sandwich Elisa assays were prepared in 96 well Maxisorp plates (NUNC) by coating with 100 μl in each well of unlabeled capture TBL1 antibody (Santa Cruz Biotech) diluted to a final concentration of 2 ug/ml in carbonate/bicarbonate coating buffer, and incubated 18 hours at 4° C. The plate is washed 3 times with PBS 0.05% Tween-20, and blocked with 200 μl of PBS 1% BSA for 1 h at RT, HCT15 cell lysates (100 μl), was added to each well to capture the TBL1:β-catenin complex. The sealed plate was incubated for 2 h at RT, washed 3 times with PBS 0.05% Tween-20, and then the complex and the recombinant protein was detected by antibody against β-catenin (Cell Signaling Tech) and secondary antibody HRP-conjugated (Sigma). Signal was detected by addition of ABTS HRP substrate

(SIGMA), and read with a microplate reader set to 405 nm.

To determine the amount of β-catenin captured, a standard curve was created using recombinant β-catenin (100 fg to 100 ng, Abnova) added instead of cell lysates in the above procedure. Intensity of signal was compared to values generated using test article treated lysates and approximate amount of bound beta catenin determined.

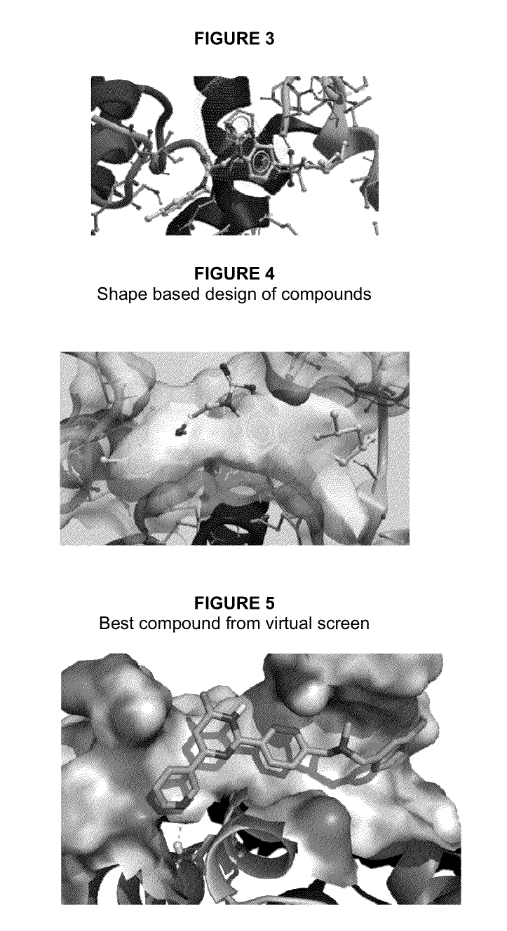

FIG. 3 shows results of this experiment that indicate Compound 1 is able to interfere with TBL1 and β-catenin interaction, with an IC50 of 10-20 nM.

### Example 2

### Screening Assay to Identify Small Molecule Inhibitors of TBL1 and β-Catenin Binding

A cell free screening assay can be employed to identify inhibitors of TBL1 and β-catenin interaction. A GST-beta catenin fusion protein (CTNNB1 amino acids 1-781 fused at the N-terminus with GST, Sino Biological) is immobilized onto a 384 well glutathione coated microtiter plate (Thermo Scientific). GST-beta catenin was diluted in PBS and 100 microliter added to each well to coat surface. The plate was washed with PBS with 0.5% Tween 20, and increasing concentrations of test agent added up to 10 micromolar. Purified TBL1 protein (100 microliters of a 1 μg/ml solution) was added to each well in 50 mM TrisHCl pH 7.4, 10 mM MgCl₂. An antibody against TBL labeled with CY5 fluorescent dye is added and incubated for 2 hours. Then, wells are washed with PBS 0.05% Tween-20 to remove unbound TBL1-CY5, and read on a fluorescent plate reader. Test agents that are able to block the binding of TBL1 to beta catenin are identified by a dose dependent decrease in signal with increasing amounts of test agent added.

Additionally, Compound 1 can be modified to contain a functional group, such as biotin, capable of being attached to a solid support and test agents identified that disrupt the binding of immobilized Compound 1 to TBL1 protein. High affinity binding Elisa 96 well plate (Maxisorp, Nunc) was coated with 100 μl to each well of full length recombinant human TBL1 or full length recombinant human β-catenin (ABNOVA) diluted to a final concentration of 1 μg/ml in carbonate/bicarbonate coating buffer, sealed to prevent evaporation, and incubated O/N at 4° C. After washing the plate 3 times with PBS 0.05% Tween-20, and blocking the plate with 200 μl of PBS 1% BSA each well for 1 h at RT, 100 μl of biotinylated Compound 1 was added to each well, at concentration in the range of 0.1 nM to 10 μM, diluted in binding buffer (50 mM TrisHCl pH 7.4, 10 mM MgCl₂). The sealed plate was incubated for 2 h at room temperature, washed 3 times with PBS 0.05% Tween-20, and then the bounded compound was detected by streptavidin-HRP conjugated antibody (Cell Signaling Tech) diluted in blocking buffer. Elisa was developed by addition of ABTS HRP substrate (SIGMA), and the optical density (OD) for each well was read with a microplate reader set to 405 nm. The results show that BC2059 is able to bind TBL1, but not β-catenin, with an IC50 of 20 nM at the equilibrium. To validate the saturation binding assay, homologous and heterologous competitive binding experiment were performed in an in vitro system.

Briefly, for homologous competitive assay, a single concentration of 50 nM of biotinylated compound was added to the wells containing TBL1 recombinant protein, in the presence of various concentrations of unlinked compound (range of 0.1 nM to 10 μM) diluted in binding buffer. The sealed plate was incubated for 2 h at RT, and then processed as described above. For the heterologous competitive binding assay, 75 nM of β-catenin was used in place of the biotinylated compound, and the bounded protein was detected by anti-β-catenin antibody (Cell Signaling Tech) followed by secondary HRP-conjugated antibody.

### Example 3

### Computational Identification of Test Compounds Related to Compound 1

The design of various new compounds was done by docking of Compound 1 in to the lateral pocket of TBL1 using GOLD technique (Jones et al., 1995). For each docking, multiple poses were generated and ranked by the GOLDSCORE scoring function. Other similar programs area available and can be used such as UNITY, FlexX, DOCK, CATALYST, and SANDOCK. To predict the interaction sites on the surface of protein, we employed the ICM Optimal Docking Area (ODA) method and predicted the optimal surface with the lowest docking desolvation energy for the SMRT and Compound 1 complex structure. The key interactions of Compound 1 with the TBL1 site confirm that the any modification of anthracine ring alters the interface domain of TBL1, and that the central oxime moiety is oriented to form an H-bonding interaction. Using this information, structural analogs of Compound 1 can be selected that had provide better binding energy. Binding affinity was measured after immobilization of GST-beta catenin as in example 2. Increasing amounts of test compound were added to a series of wells and inhibition of TBL1 binding measured. Compounds that have dose responsive inhibition of beta catenin to TBL1 binding were selected. Binding affinities were compared to Compound 1, and competition experiments using compound I were used to identify test compounds that have similar or better activity than Compound 1.

### Example 4

### Computational Identification of Test Compounds Able to Inhibit TBL1 and 13-Catenin Binding

The design of various new compounds was done by docking a known compound library in to the lateral pocket of TBL1 using GOLD technique (Jones et al., 1995). For each docking, multiple poses were generated and ranked by the GOLDSCORE scoring function. Other similar programs area available and can be used such as UNITY, FlexX, DOCK, CATALYST, and SANDOCK. Critical residues were identified and changes made based on induced fit docking and complex binding energy minimization. Identified compounds can be selected and tested for ability to inhibit binding of TBL1 to beta catenin using GST fused to beta catenin and purified TBL1. Binding was measured be immobilization of GST-bet catenin in a 96 well plate. Increasing amounts of test compound were added to a series of wells and TBL1 added and allowed to bind to beta catenin at 4° C. for 18 hrs. Wells were washed to remove unbound TBL1 and remaining TBL1 detected using an antibody to TBL1 labeled with CY5 and signal detected using a fluorescent plate reader.

To predict the interaction sites on the surface of protein, we employed the ICM Optimal Docking Area (ODA) method and predicted the optimal surface with the lowest docking desolvation energy for the SMRT and Compound 1 complex structure. We used the TBL1/Compound 1 complex structure in search for novel scaffolds with ideal candidate properties. The large scale-virtual screening of ˜2 MM) libraries was performed which led to the identification of screening hits. These new series of compounds bind at the same region of Compound 1 but the increase in binding energy over Compound 1 is due to gain in πιπι interactions with Phe10 which is one of the critical residue identified using site map and such interactions were not seen with Compound 1.

### Methods of Making the Compounds

In one aspect, the invention relates to methods of making compounds useful as inhibitors of TBL1, which can be useful in the treatment of disorders of uncontrolled cellular proliferation. In a further aspect, the TBL1, The compounds of this invention can be prepared by employing reactions as shown in the following schemes, in addition to other standard manipulations that are known in the literature, exemplified in the experimental sections or clear to one skilled in the art. In one aspect, the disclosed compounds comprise the products of the synthetic methods described herein. In a further aspect, the disclosed compounds comprise a compound produced by a synthetic method described herein.

### General Methods

All routine reagents and solvents were purchased from Sigma Aldrich and used as received. They were of reagent grade, purity≧99%. Specialty chemicals and building blocks obtained from several suppliers were of the highest offered purity (always ≧95%). NMR spectroscopy was performed on a Mercury 400 MHz operating at 400 MHz, equipped with a 5 mm broadband probe and using standard pulse sequences. Chemical shifts (δ) are reported in parts-per-million (ppm) relative to the residual solvent signals. Coupling constants (J-values) are expressed in Hz. Mass spectrometry was performed on a Waters Quattro-II triple quadrupole mass spectrometer. All samples were analyzed by positive ESI-MS and the mass-to-charge ratio (m/z) of the protonated molecular ion is reported. Microwave-assisted reactions were performed on a Biotage Initiator 2.5 at various powers. Hydrogenation reactions were performed on a standard Parr hydrogenation apparatus. Reactions were monitored by TLC on Baker flexible-backed plates coated with 200 μm of silica gel containing a fluorescent indicator. Preparative TLC was performed on 20 cm×20 cm Analtech Uniplates coated with a 1000 or 2000 μm silica gel layer containing a fluorescent (UV 254) indicator. Elution mixtures are reported as v:v. Spot visualization was achieved using UV light. Flash chromatography was performed on a Teledyne Isco CombiFlash RF 200 using appropriately sized Redisep Rf Gold or Standard normal-phase silica or reversed-phase C-18 columns. Crude compounds were adsorbed on silica gel, 70-230 mesh 40 Å (for normal phase) or Celite 503 (for reversed-phase) and loaded into solid cartridges. Elution mixtures are reported as v:v.

### Example 5

### Synthesis of Formula I Compounds (Prophetic)

Compounds of Formula I 4-1H-pyrazol-pyrimidin series of compounds) prepared using the procedures outlined in Scheme I below.

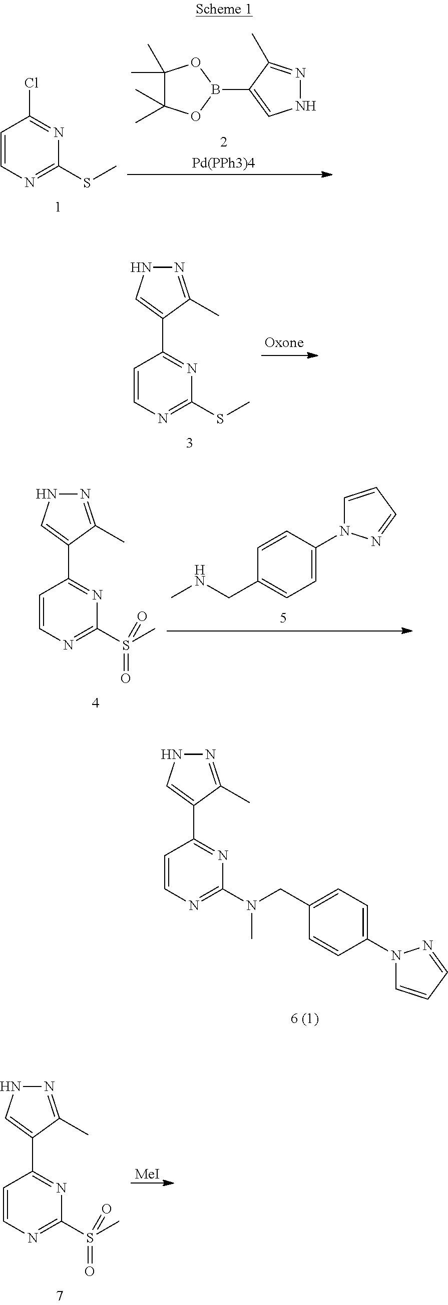

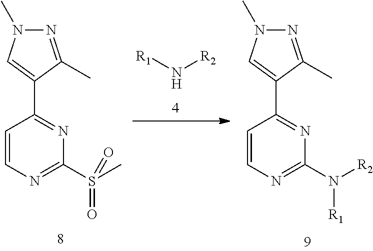

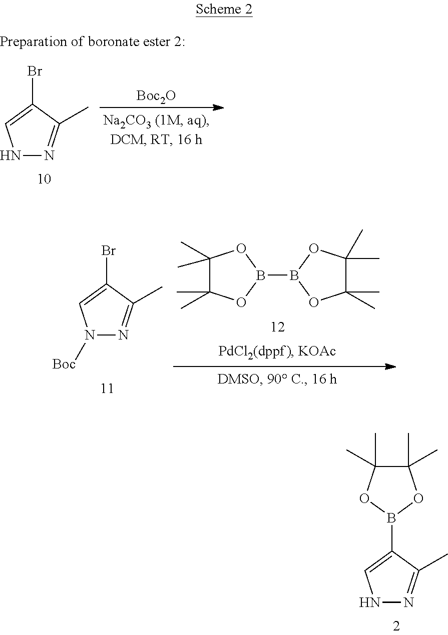

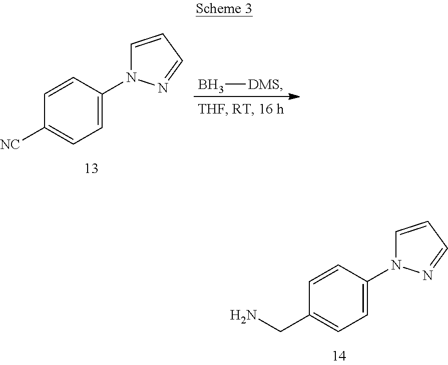

Compound 11 is prepared from compound 10 by reacting (1 eq), Boc₂O (1.5 eq), Na₂CO₃ (2 eq) in DCM at RT for 16 h. After work up and the NMR showed characteristic peaks and was used as such for the next step. In subsequent step compound II (1 eq), and 12 (1.1 eq) in presence of PdCl₂(dppf) (0.05 eq), KOAc (3 eq), DMSO was heated to 90° C., 16 h. After column purification LCMS showed 50% of desired mass of compound 11. Key intermediate 14 was prepared was prepared by reacting (1 eq), BH₃-DMS (3 eq), THF, RT, 16 h. After work up LCMS showed 96% purity of the compound 14.

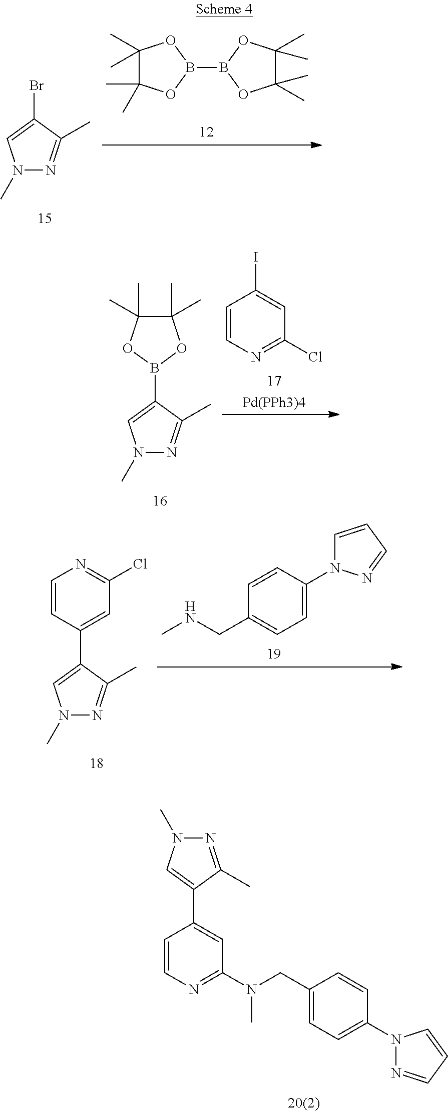

### Compound 15

(1 eq), n-BuLi (1.2 eq), THF, -78° C., 5 min; 12 (1.2 eq) in THF, −78° C. to RT, 1 h. Crude LCMS showed 42% of desired mass of compound 16. The crude was used as such for the next step where compound 17 (1 eq), 16 crude (1.2 eq), Pd(PPh₃)₄ (0.05 eq), Na₂CO₃ (3 eq), toluene/EtOH/H₂O was heated to 90° C. Work up and purification provided the desired producted 18. The compound 18 under similar conditions were reacted with 19 to obtain the final product 20(6).

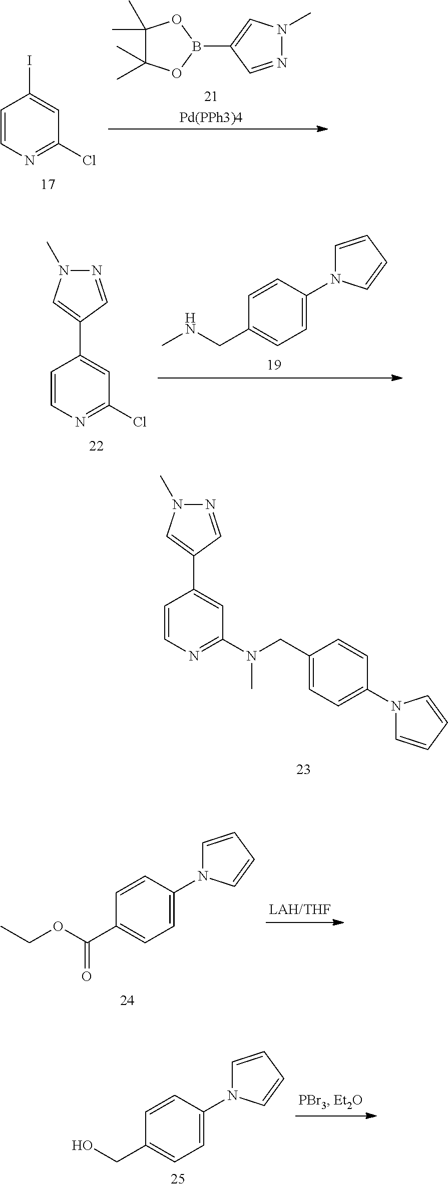

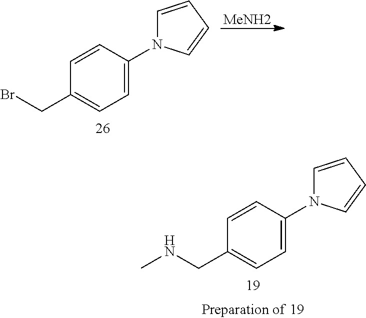

### 2-chloro-4-iodopyridine

(1 eq), compound 21 (1.2 eq) in presence of Pd(PPh₃)₄ (0.05 eq), Na₂CO₃ (3 eq) and toluene/EtOH/H₂O, 90° C. was refluxed for 24 hrs. The obtained compound 22 was subsequently reacted with compound 19 to obtain the desired product 23 in 36% yields.

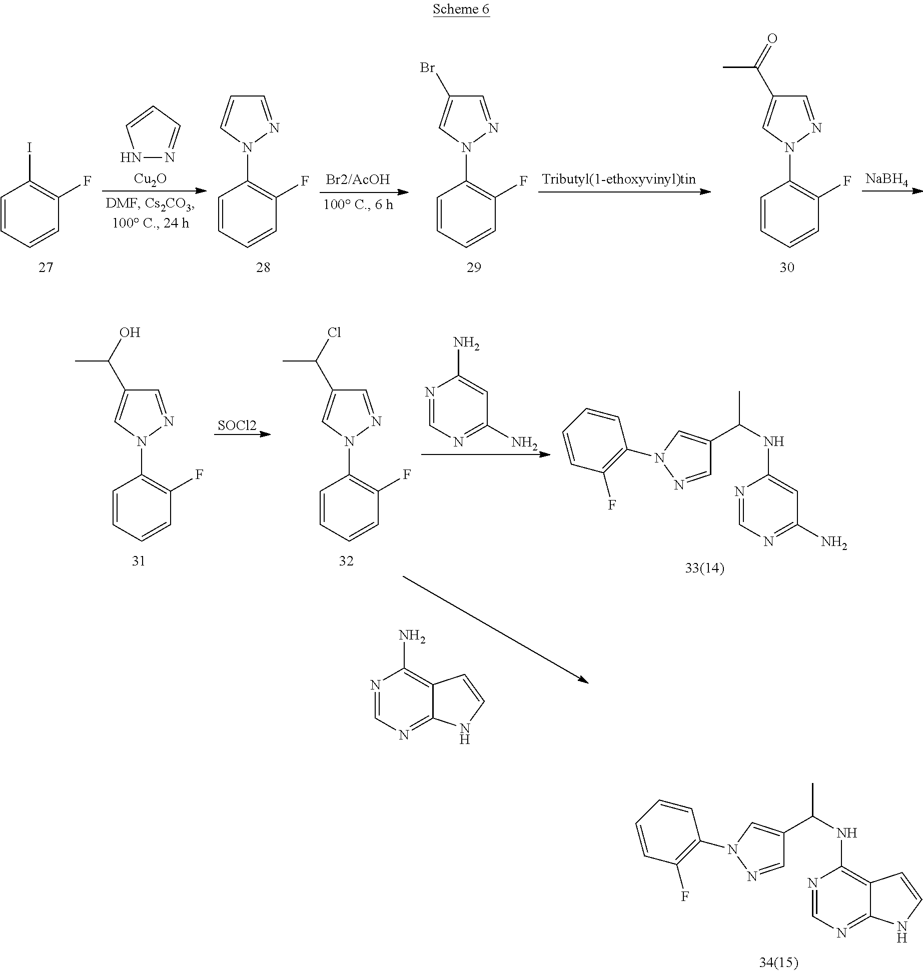

Pyrazole (1 eq), 27 (1.5 eq), Cu₂O (0.1 eq), Cs₂CO₃ (2 eq) in DMF was heated to 100° C., 16 h. After column purification LCMS showed 85% purity. NMR complies complies with the compound 28. In sequential reactions shown in schemes 6 provided the desired compound 33 and 34 in 26 and 35% yields.

### Example 6

### Synthesis of Formula II Compounds (Prophetic)

Compounds of Formula II (2-phenylpyrimidin-4(3H)-one series of compounds) may be prepared using the procedures outlined in Scheme II below. No representation is being made that the synthesis has been performed.

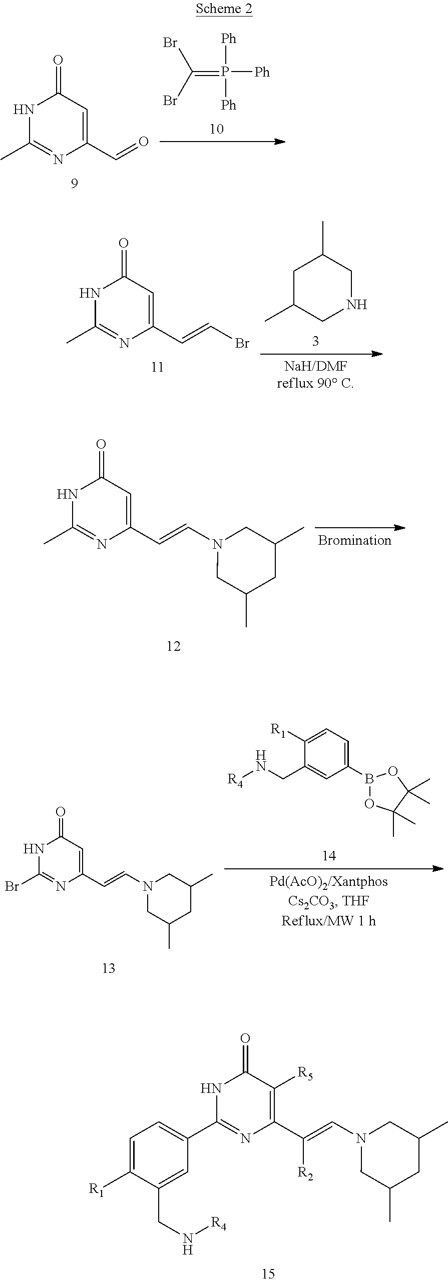

### Example 7

### Synthesis of Formula III Compounds (Prophetic)

Compounds of Formula III (N-((1H-pyrazol-4-yl)methyl)-9H-purin-6-amine series of compounds) may be prepared using the procedures outlined in Scheme III below. No representation is being made that the synthesis has been performed.

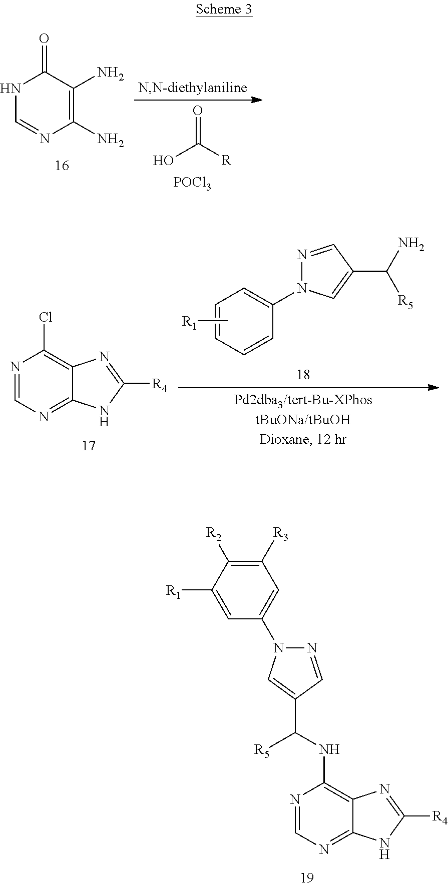

### Example 8

### Synthesis of Formula IV Compounds (Prophetic)

Compounds of Formula IV (2-(1H-indol-1-yl)-N-(2H-1,2,3-triazol-4-yl)acetamide series of compounds) may be prepared using the procedures outlined in Schemes IV and V below. No representation is being made that the synthesis has been performed.

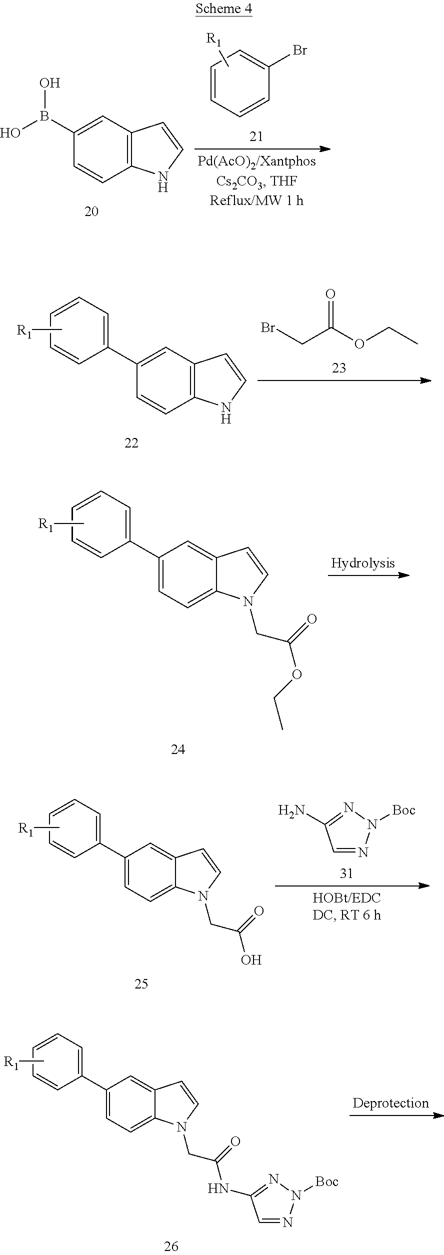

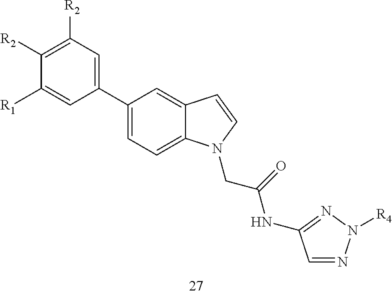

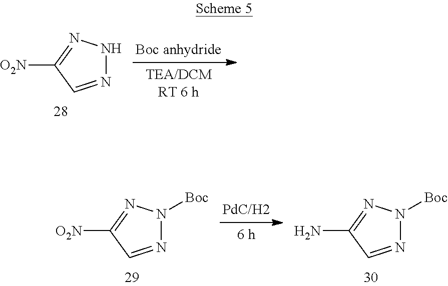 [SMILES: O=[N+]([O-])c1cn[nH]n1 ; CC(C)(C)OC(=O)n1ncc([N+](=O)[O-])n1 ; CC(C)(C)OC(=O)n1ncc(N)n1]

### Methods of In Vitro Screening

### Sandwich ELISA Protocol

HCT 15 and HCT116 cells (colon cancer cell line with stable expression of Beta-catenin) were seeded at 70% confluency in RPMI 10% FBS (Gibco). After 24 h the medium was changed with fresh one. TBL1-specific compound (BC2059, 19, 29) was added at concentration in the range of 0.3 nM to 3 μM for 6H 37° C. The cells were lysed in RIPA buffer containing protease inhibitors mix, and the total protein concentration was determined by Quick Start Bradford Protein assay kit (Bio-Rad). To prepare the high affinity binding Sandwich Elisa plate, 96 well Maxisorp (NUNC) was coated with 100 μl to each well of unlabeled capture TBL1 antibody (Santa Cruz Biotech) diluted to a final concentration of 2 ug/ml in carbonate/bicarbonate coating buffer, sealed to prevent evaporation, and incubated 0/N at 4° C. After washing the plate 3 times with PBS 0.05% Tween-20, and blocking the plate with 200 μl of PBS 1% BSA each well for 1 h at RT, 100 μl of HCT15 cell lysates, containing equal amount of total proteins, were added to each well containing TBL1 antibody, to capture TBL1:β-catenin complex. To determine the amount of β-catenin captured, we prepared a standard curve with recombinant β-catenin (100 fg to 100 ng), diluted in carbonate/bicarbonate coating buffer. The sealed plate was incubated for 2 h at RT, washed 3 times with PBS 0.05% Tween-20, and then the complex and the recombinant protein were detected by antibody against β-catenin (Cell Signaling Tech) and secondary antibody HRP-conjugated (Sigma) diluted in blocking buffer. Elisa was developed by addition of ABTS HRP substrate (SIGMA), and the optical density (OD) for each well was read with a microplate reader set to 405 nm.
Saturation Binding assay and Competition Binding Assay with Linkered BC2059 Protocol.

To determine whether the ability of BC2059 to interfere with TBL1:β-catenin complex formation is due to direct interaction between TBL1 or β-catenin and the compound, we performed saturation binding assays in a in vitro system. High affinity binding Elisa 96 well plate (Maxisorp, Nunc) was coated with 100 ul to each well of full length recombinant human TBL1 or full length recombinant human β-catenin diluted to a final concentration of 1 ug/ml in carbonate/bicarbonate coating buffer, sealed to prevent evaporation, and incubated O/N at 4° C. After washing the plate 3 times with PBS 0.05% Tween-20, and blocking the plate with 200 μl of PBS 1% BSA each well for 1 h at RT, 100 μl of biotinylated compound was added to each well containing the recombinant proteins, at concentration in the range of 0.1 nM to 10 μM, diluted in binding buffer (50 mM TrisHCl pH 7.4, 10 mM MgCl2). The sealed plate was incubated for 2 h at RT, washed 3 times with PBS 0.05% Tween-20, and then the bounded compound was detected by streptavidin-HRP conjugated antibody (Cell Signaling Tech) diluted in blocking buffer. Elisa was developed by addition of ABTS HRP substrate (SIGMA), and the optical density (OD) for each well was read with a microplate reader set to 405 nm. To validate the saturation binding assay, homologous and heterologous competitive binding experiment were performed in an in vitro system. Briefly, for homologous competitive assay, a single concentration of 50 nM of biotinylated compound was added to the wells containing TBL1 recombinant protein, in the presence of various concentrations of unlinked compound (range of 0.1 nM to 10 uM) diluted in binding buffer. The sealed plate was incubated for 2 h at RT, and then processed as described above. For the heterologous competitive binding assay, 75 nM of β-catenin was used in place of the biotinylated compound, and the bounded protein was detected by anti-β-catenin antibody (Cell Signaling Tech) followed by secondary HRP-conjugated antibody.

TABLE 5
Lead compounds TBL1 inhibition and cell screening data

| Compound | EC₅₀ μM | EC₅₀ μM | EC₅₀ μM |
|---|---|---|---|
| No: | TBL1 | HCT116 CELLS | TBL1: β-Catenine |
| 9 (29) | 0.22 | 0.0843 | 0.21 |
| 10 (16) | 0.28 | NA | NA |
| 11 (22) | 0.29 | NA | NA |
| 12 (34) | 102.1 | NA | NA |
| 13 (35) | 0.024 | NA | NA |
| 16 (19) | 0.054 | 0.127 | 0.41 |

NA: Not Available

### Sequence Listing

Sequence 1 is an amino acid sequence of TBL1X isoform A protein.
Sequence 2 is an amino acid sequence of TBL1X isoform B protein.
Sequence 3 is an amino acid sequence of TBL1Y protein.
Sequence 4 is an amino acid sequence of TBL1XR1 protein.

The ASCII text file “Sequence.txt” created on Nov. 12, 2012, having the size of 20 KB, is incorporated by reference into the specification.

What is claimed is:

1. A method of treating and/or preventing a beta-catenin related disorder comprising administering to a patient in need thereof a therapeutically effective amount of an agent that binds to a lateral groove of TBL1 protein having the sequence selected from the group consisting of SEQ ID NO: 1-4, thereby preventing binding of beta-catenin to said lateral groove.

2. The method of claim 1, wherein said lateral groove of TBL1 protein is defined by residues 32 to 57 of SEQ ID NO: 1.

3. The method of claim 1, wherein said agent is selected from the group consisting of a small molecule, a peptide, or a mimetic, wherein said small molecule has a molecular weight of no more than 1000 Daltons.

4. The method of claim 1, wherein said beta-catenin related disorder comprises cancer.

5. The method of claim 4, wherein said cancer comprises colon cancer.

6. An agent for treating and/or preventing a beta-catenin related disorder, wherein said agent upon administration to a patient in need thereof binds to a lateral groove of TBL1 protein having the sequence selected from the group consisting of SEQ ID NO: 1-4, thereby preventing binding of beta-catenin to said lateral groove.

7. The agent of claim 6, wherein said lateral groove of TBL1 protein is defined by residues 32 to 57 of SEQ ID NO: 1.

8. The agent of claim 6 having the following structure: 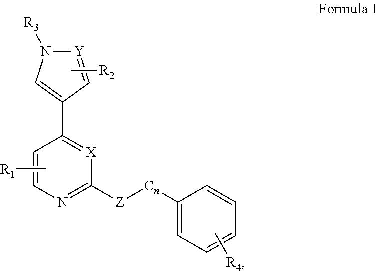
wherein
X and Y are selected from N and C;
R₁ is selected from H, halogen, CF₃, OCF₃, CN, SO₂CH₃, (CH₃)₂CN, SO₂NH₂, SO₂NCH₃, SO₂(CH₃)₂, SON(CH₃)₂, and C═ON(CH₃)₂;
R₂ is selected from H, CH₃, CH₂CH₃, F, and Cl;
R₃ is selected from H, —CH₃, C₁-C₆ alkyl, C₁-C₆ cycloalkyl, and C₁-C₆ hetero-cycloalkyl;
R₄ is selected from H, halo, OCH₃, CH₃, OH, NH₂, C₁-C₆ cycloalkyl, and C₁-C₆ hetero-cycloalkyl;
R₅ is selected from —CH₃ and halo;
Z is selected from —NH, O, S, N—CH₃, —NCH₂CH₃, and SO₂; and
n is 1 or 2;
or a pharmaceutically acceptable salt thereof.

9. The agent of claim 6 having the following structure: 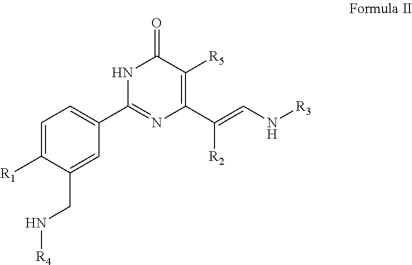
wherein
X is selected from N and C;
R₁ is selected from H, halogen, CF₃, OCF₃, CN, SO₂CH₃, (CH₃)₂CN, SO₂NH₂, SO₂NCH₃, SO₂(CH₃)₂, SON(CH₃)₂, and C═ON(CH₃)₂;
R₂ is selected from H, CH₃, CH₂CH₃, F, and Cl;
R₃ is selected from H and C₁-C₆ alkyl, Ar and Ar¹;
wherein Ar is phenyl substituted with 0-3 groups independently selected from cyano, C₁-C₆ alkyl, C₁-C⁶ haloalkyoxy, C₁-C₆ haloalkyl, C₁-C₆ polyhaloalkyl, C₁-C₆ cyanoalkyl, SO₂CH₃, SO₂CH₂CH₃, SO₂N(CH₃)₂, SO₂NH₂, SO₂NH—CH₃, SO₂—NHCF₃, SO₂NHCH₂CF₃, C₁-C₃ alkyl, C₁-C₃ alkylamine, and C₁-C₃ dialkylamino,
wherein Ar¹ is monocyclic heteroaryl substituted with 0-3 groups independently selected from halo, cyano, C₁-C₆ alkyl, C₁-C₆ haloalkyoxy, C₁-C₆ haloalkyl, and C₁-C₆ polyhaloalkyl, C₁-C₆ cyanoalkyl, SO₂CH₃, SO₂CH₂CH₃, SO₂N(CH₃)₂, SO₂NH₂, SO₂NH—CH₃, SO₂—NHCF₃, SO₂NHCH₂CF₃, C₁-C₃ alkyl, C₁-C₃ alkylamine, and C₁-C₃ dialkylamino;
R₄ is selected from H, CH₃, C═OCH₃, C═OCH₂—NH₂, C═OCH₂CH₃, C═OCH═CH₂, C═OCH═CH₂, C═OPr-i, and C═OCH₂OH; and
R₅ is selected from H, CH₃, and halogen,
or a pharmaceutically acceptable salt thereof.

10. The agent of claim 6 having the following structure: 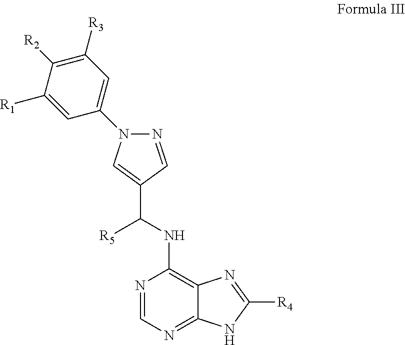
wherein
R₁ and R₃ are independently selected from hydrogen, halogen, and C₁-C₆ alkyl;
R₂ is a five-membered or six-membered C₃-C₆ heterocycle substituted with 0-3 groups selected from halogen, cyano, C₁-C₆ alkyl, C₁-C₆ haloalkyoxy, C₁-C₆ haloalkyl, and C₁-C₆ polyhaloalkyl;
R₄ is H, CH₃, halogen, CF₃, OCF₃, CN and —OCH₃;
R₅ is selected from H, CH₃, and halogen,
or a pharmaceutically acceptable salt thereof.

12. The agent of claim 6 having the following structure: 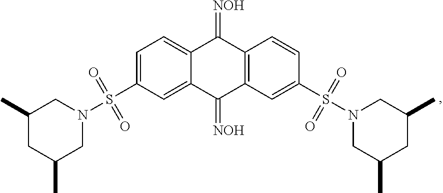
or a pharmaceutically acceptable salt thereof.

13. A pharmaceutical composition comprising the agent of claim 6 and a pharmaceutically acceptable excipient.

14. A method of identifying an agent capable of modulating a transducin beta-like protein 1 (TBL1) activity comprising:
a) contacting a biological sample comprising an activator of TBL1 with a test agent; and
b) determining whether said test agent inhibits binding of said activator of TBL1 to TBL1;
wherein if said test agent inhibits said binding, then said test agent is capable of modulating TBL1 activity.

15. The method of claim 14, wherein said TBL1 is selected from the group consisting of Transducin (beta)-like 1X-linked (TBL1X), Transducin (beta)-like 1Y-linked (TBL1Y) and Transducin (beta)-like R1-linked TBLR1 proteins.

16. The method of claim 14, wherein the inhibition of binding in step (b) is measured by determining physical association of TBL1 and said activator.

17. The method of claim 14, wherein the inhibition of binding in step (b) is measured by determining the level or the stability of said activator.

18. The method of claim 14, wherein said activator is beta-catenin.

19. The method of claim 14, wherein said activator is a beta-catenin related protein.

20. The method of claim 14, further comprising step (c) of determining whether said test agent binds to a TBL1 lateral groove.

21. A method of identifying an agent capable of modulating a transducin beta-like protein 1 (TBL1) activity comprising conducting a virtual screening of a library of test compounds, whereby said virtual screening is capable of predicting whether a test compound is able to bind to TBL1, wherein a test compound which is able to bind to TBL1 is identified as an agent capable of modulating a TBL1 activity.

22. The method of claim 21, wherein said library is a physical library of small molecules and peptides.

23. The method of claim 21, wherein said library is a virtual library of small molecules and peptides.

24. The method of claim 21, wherein said virtual screening is capable of predicting whether a test compound is able to form a hydrogen bond at position 35 of TBL1X.
